(19) United States
(12) Patent Application Publication
(10) Pub. No.: US 2023/0065606 A1
(43) Pub. Date: Mar. 2, 2023

(54) CAPILLARY ELECTROPHORESIS METHODS FOR VIRAL VECTOR SEPARATION, ANALYSIS, CHARACTERIZATION AND QUANTIFICATION

(71) Applicant: DH Technologies Development Pte. Ltd., Singapore (SG)

(72) Inventors: Tingting Li, Yorba Linda, CA (US); Fang Wang, Chino Hills, CA (US); Handy Yowanto, Walnut, CA (US)

(21) Appl. No.: 17/792,084

(22) PCT Filed: Jan. 13, 2020
(86) PCT No.: PCT/IB2021/050235

§ 371 Date: Jul. 11, 2022

Related U.S. Application Data
(60) Provisional application No. 62/960,282, filed on Jan. 13, 2020.

(51) Int. Cl. G01N 27/447; G01N 33/569; G01N 33/53

(52) U.S. Cl. CPC G01N 27/44795; G01N 33/56983; G01N 33/5308

(57) ABSTRACT

The disclosure provides methods for analyzing Adeno-Associated Vims (AAV) vectors. More particularly, the disclosure relates to capillary electrophoresis methods for separating, characterizing, and quantifying AAV vectors as having a full length exogenous polynucleotide, having an exogenous polynucleotide fragment, or lacking any exogenous polynucleotide. In one method, the analysis uses capillary isoelectric focusing (cIEF) on a sample comprising viral vectors containing exogenous polynucleotide under conditions and for a duration sufficient to separate the viral vectors based on the isoelectric point (pI) of each viral vector.

### RELATED U.S. APPLICATIONS

This application claims the benefit of priority from U.S. Provisional Application Ser. No. 62/960,282, filed on Jan. 13, 2020, the entire contents of which is hereby incorporated by reference.

### BACKGROUND

Viral vectors, such as Adeno-Associated Virus (AAV) vectors are a common molecular biology tool for gene delivery, and show promise in gene therapy among other applications. A limitation of this technology is that synthesizing vectors (e.g., AAVs) typically results in a heterogeneous sample in which some vectors will contain only a fragment of a target transgene (“partial vector”) or entirely lack a target transgene (“empty vector”). Partial and empty vectors are considered impurities (e.g., “contaminant” vectors) that can negatively impact downstream viral transduction by competing with properly packaged vectors (i.e. vectors having a full encapsulated transgene) for vector binding sites on target cells. Further, these contaminating vectors can increase immunogenicity of a viral vector product.

During viral vector research and development, it is therefore advantageous to identify these contaminant vectors. Techniques to identify full and empty vectors that include analytical ultracentrifugation, electron microscopy, spectrophotometry and anion-exchange HPLC, provide minimal resolution and sensitivity, such as the inability to determine partial vectors, and poor reproducibility. These techniques are also time-consuming and require a large sample volume.

Described herein are novel capillary electrophoresis techniques for separating, characterizing, and quantifying full, partial, and empty viral vectors that overcome the disadvantages of other analytical technologies.

### SUMMARY OF THE DISCLOSURE

The disclosure generally provides a method of separating viral vectors based on exogenous polynucleotide content. In an aspect, the disclosure provides a method of separating viral vectors in a sample by (a) performing capillary isoelectric focusing (cIEF) on a sample comprising viral vectors containing exogenous polynucleotide under conditions and for a duration sufficient to separate the viral vectors based on the isoelectric point (pI) of each viral vector; (b) generating a readout of cIEF results; and (c) analyzing the readout to identify viral vectors based on exogenous polynucleotide content. The method characterizes the viral vectors as having a full length exogenous polynucleotide, an exogenous polynucleotide fragment, or lacking any exogenous polynucleotide. In one aspect, viral vectors having the full length exogenous polynucleotide have an alternate pI relative to the viral vectors having an exogenous polynucleotide fragment or viral vectors lacking any exogenous polynucleotide.

In some aspects, the exogenous polynucleotide comprises a transgene, promoter and ITR sequence. In some aspects, the viral vectors is an adeno-associated virus vector (AAV).

In some aspects, the readout of cIEF results is an electropherogram or a separation scan that employs whole-column imaging detection (WCID) technology that allows cIEF processes to be imaged in real time.

In some aspects, the cIEF method includes (a) loading a sample comprising viral vectors into a channel or a capillary; (b) fluidly connecting a first end of the channel or capillary to an acidic solution; (c) fluidly connecting a second end of the channel or capillary to a basic solution; and (d) applying an electric voltage to the channel or capillary to induce/form a pH gradient in the channel or capillary. In certain aspects, the channel or capillary is loaded (i.e., filled completely) with sample that, in some embodiments, comprises 2-4 μL of viral vectors, depending on concentration. Thus, in accordance with some example embodiments the sample may comprise one or more viral vectors, and further comprise gel or gel matrix, a solubilizer (e.g., urea, glycerol, etc.), carrier ampholyte, anodic stabilizer, cathodic stabilizer, and pI markers.

The present disclosure further provides a method of quantifying viral vectors based on exogenous polynucleotide content by separating the species and measuring the peak area of the corresponding peaks in the electropherogram. The method can include (a) performing capillary isoelectric focusing (cIEF) on a sample comprising viral vectors containing exogenous polynucleotide under conditions and for a duration sufficient to separate the viral vectors based on the isoelectric point (pI) of each viral vector; (b) generating a readout of cIEF results; and (c) analyzing the readout to identify viral vectors based on exogenous polynucleotide content. The method characterizes the viral vectors as having a full length exogenous polynucleotide, an exogenous polynucleotide fragment, or lacking any exogenous polynucleotide. In one aspect, viral vectors having the full length exogenous polynucleotide have an alternate/different pI relative (either lower or higher) to the viral vectors having an exogenous polynucleotide fragment or viral vectors lacking any exogenous polynucleotide.

In some aspects, the exogenous polynucleotide comprises a transgene, promoter or ITR sequence. In some aspects, the viral vectors is an adeno-associated virus vector (AAV).

In some aspects, the readout of cIEF results is an electropherogram or a separation scan.

In some aspects, the cIEF method includes (a) loading a sample comprising viral vectors into a channel or a capillary; (b) fluidly connecting a first end of the channel or capillary to an acidic solution; (c) fluidly connecting a second end of the channel or capillary to a basic solution; and (d) applying an electric voltage to the channel or capillary to cause the formation of a pH gradient in the channel or the capillary. In certain aspects, the channel or capillary is loaded with 2-4 μL of vector material that comprises at least a portion of the sample.

In another aspect, the present disclosure provides a method of separating viral vectors in a sample by (a) performing capillary electrophoresis (CE) of a sample comprising viral vectors containing exogenous polynucleotide under conditions and for a duration sufficient to separate the viral vectors based on the charge heterogeneity or isoelectric point (pI) of each viral vector; (b) generating a readout of CE results; and (c) analyzing the readout to identify viral vectors based on exogenous polynucleotide content. The method characterizes the viral vectors as having a full length exogenous polynucleotide, an exogenous polynucleotide fragment, or lacking any exogenous polynucleotide.

In some aspects, the exogenous polynucleotide comprises a transgene. In some aspects, the viral vectors is an adeno-associated virus vector (AAV).

In some aspects, the readout of CE results is an electropherogram or a separation scan.

In some aspects, the CE method includes (a) loading a sample comprising viral vectors into a channel or a capillary; (b) fluidly connecting a first end of the channel or capillary to an anodic terminal; (c) fluidly connecting a second end of the channel or capillary to a cathodic terminal; and (d) applying an electric voltage to the channel or capillary. The CE method can include, but is not limited to, capillary isoelectric focusing (cIEF), capillary zone electrophoresis (CZE), capillary gel electrophoresis (CGE), capillary isotachophoresis, micellar electrokinetic capillary chromatography and capillary electrokinetic chromatography. In some embodiments, the anodic terminal can comprise an acidic solution. In some embodiments, the cathodic terminal can comprise a basic solution.

The present disclosure further provides a method of quantifying viral vectors based on exogenous polynucleotide content. The method can include (a) performing capillary electrophoresis (CE) of a sample comprising viral vectors containing exogenous polynucleotide under conditions and for a duration sufficient to separate the viral vectors based on the charge heterogeneity or isoelectric point (pI) of each viral vector; (b) generating a readout of CE results; and (c) analyzing the readout to identify viral vectors based on exogenous polynucleotide content. The method characterizes the viral vectors as having a full length exogenous polynucleotide, an exogenous polynucleotide fragment, or lacking any exogenous polynucleotide.

In some aspects, the exogenous polynucleotide comprises a transgene, promoter and ITR sequence. In some aspects, the viral vectors is an adeno-associated virus vector (AAV).

In some aspects, the readout of CE results is an electropherogram or a separation scan.

In some aspects, the CE method includes (a) loading a sample comprising viral vectors into a channel or a capillary; (b) fluidly connecting a first end of the channel or capillary to an anodic terminal; (c) fluidly connecting a second end of the channel or capillary to a cathodic terminal; and (d) applying an electric voltage to the channel or capillary. The CE method can include, but is not limited to, capillary isoelectric focusing (cIEF), capillary zone electrophoresis (CZE), and capillary gel electrophoresis (CGE). In some aspects, the anodic terminal can comprise an acidic solution. In some embodiments, the cathodic terminal can comprise a basic solution.

Also disclosed herein is a system to carry out the above methods.

### BRIEF DESCRIPTION OF THE DRAWINGS

FIG. 1 illustrates examples of a viral capsid having a full exogenous polynucleotide, an empty viral capsid, and viral capsids containing an exogenous polynucleotide fragment or a contaminant fragment.

FIG. 2 illustrates an exemplary embodiment of a system to separate viral vectors.

FIG. 3A depicts initial instrument parameters for a cIEF method according to some embodiments of the present disclosure.

FIG. 3B depicts initial UV detector parameters for a cIEF method according to some embodiments of the present disclosure.

FIG. 3C depicts instrumental conditioning parameters for a cIEF method according to some embodiments of the present disclosure.

FIG. 3D depicts instrumental separation parameters for a cIEF method according to some embodiments of the present disclosure.

FIG. 3E depicts instrumental shutdown parameters for a cIEF method according to some embodiments of the present disclosure.

FIG. 4 illustrates cIEF separation results of a first AAV sample enriched with empty viral capsids and a second AAV sample enriched with full viral capsids.

FIG. 5A illustrates cIEF and AEX-HPLC separation results of a third AAV sample.

FIG. 5B is an enlarged view of the results shown in FIG. 5A.

FIG. 6A illustrates cIEF separation results of a fourth AAV sample enriched with empty viral capsids and a fifth AAV sample enriched with full viral capsids.

FIG. 6B is an enlarged view of the results shown in FIG. 6A.

FIG. 7 illustrates cIEF separation results of a sixth AAV sample using a mixture of wide and narrow range pH ampholytes or only wide range pH ampholytes.

FIG. 8 illustrates cIEF separation results from five runs of a sixth AAV sample.

FIG. 9 illustrates CZE separation results

FIG. 10 illustrates CZE separation results

### DETAILED DESCRIPTION

Described herein are methods of separating and/or quantifying viral vectors based on their exogenous polynucleotide content using capillary electrophoresis (CE). “Capillary electrophoresis” or “CE” refers to a family of related techniques that employ a capillary to separate large and small molecules, based on size and charge, by their different rates of migration in an electric field. CE techniques include capillary zone electrophoresis (CZE), capillary isoelectric focusing (cIEF), capillary gel electrophoresis (CGE), isotachophoresis, micellar electrokinetic capillary chromatography and capillary electrochromatography. As used herein, the term “capillary” refers to a channel, tube, or other structure capable of supporting a volume of separation medium for performing electrophoresis. Capillary geometry can vary and includes structures having circular, rectangular, or square cross-sections, channels, groves, plates, etc. that can be fabricated by technologies known in the art. Capillaries of the present disclosure can be made of materials such as, but not limited to, silica, fused silica, quartz, silicate-based glass such as borosilicate glass, phosphate glass, or alumina-containing glass, and other silica-like materials. In some embodiments the methods may be adapted and used in any generally known electrophoresis platform such as, for example, electrophoresis devices comprising single or multiple microfluidic channels, etched microfluidic capillaries, as well as slab gel and thin-plate gel electrophoresis.

As used herein, a “viral vector” refers to a viral construct (e.g., viral molecule comprising one or more of DNA, RNA, or protein) used as a vehicle to harbor or carry foreign genetic material (i.e., an exogenous polynucleotide) into another cell where it can be replicated and/or expressed. Exemplary viral vectors include retroviruses, lentiviruses, adenoviruses, adeno-associated viruses (AAV), and hybrids that are genetically modified to have qualities of more than one vector.

Generally, CE and in particular cIEF utilizes a capillary with an optical viewing window, a high voltage power supply, two electrode assemblies, two buffer reservoirs, and a detector. One buffer reservoir contains an acidic solution that provides H⁺ ions into the capillary, while one buffer reservoir contains a basic solution that provides OH⁻ions into the capillary. A first end of the capillary is fluidly connected to the basic solution while the second end is fluidly connected to the acidic solution. In some embodiments, the same type of buffer can be utilized in each of the two buffer reservoirs as long as one reservoir is capable of operating as an anode terminal and the other of the two reservoirs is capable of operating as a cathode terminal. Samples or analytes can be injected into the capillary via electro-injection or pressure injection. In some embodiments, a capillary is loaded with 2-4 μL of vector material that comprises a portion of the sample undergoing analysis. In some embodiments, the buffer solution need not alternatively be an acidic or basic solution. In some instances, the same buffer solution can be provided to the first and second ends of the capillary so long as it is able to provide H⁺ and OH⁻ ions. Generally, a low sample volume of less than 5 uL is consumed in the analysis.

Sample migration is initiated by applying an electric field, which is supplied to the electrode assemblies from the power supply. Sample components or analytes of interest separate as they migrate due to electroosmotic flow and their electrophoretic mobility, forming a gradient of separated components or analytes, and are subsequently detected near the outlet end of the capillary. Detector output is processed by an integrator, microprocessor, or computer. The resulting CE separation data is displayed as an electropherogram, which reports detector response as a function of time. In some aspects, the CE readout is an electropherogram or separation scan. Suitable CE detectors include, but are not limited to, ultraviolet (UV), UV-visible (UV-Vis), laser-induced fluorescence, native fluorescence, diode array, refractive index, electrochemical, surface plasmon resonance, Raman scattering, and mass spectrometry. Suitable CE optical detectors include, but are not limited to, CCD arrays and photomultiplier tubes.

In a particular aspect, the present disclosure relates to a method for performing CE to separate and characterize viral vectors as having a full length exogenous polynucleotide (“a full viral vector”), an exogenous polynucleotide fragment (“a partial viral vector”), or lacking any exogenous polynucleotide (“an empty viral vector”). This method can also distinguish and quantify the relative abundance of full, partial, and empty viral vectors in a sample or preparation comprising viral vectors. In some embodiments, the methods disclosed herein may be used to separate and characterize lipid nanoparticles, liposomes, virosomes, and carbon nanotubes.

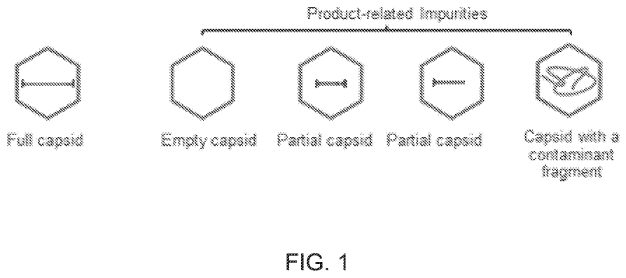

In exemplary embodiments, the viral vector comprises an AAV, and the methods disclosed herein separate and characterize AAV capsids, particles, or vectors as having a full transgene, promoter and ITR sequence, a transgene fragment, or lacking a transgene (FIG. 1). In some aspects, the methods can also distinguish AAV capsids, particles, or vectors having a contaminant or fragment thereof. As used herein, “transgene” refers to a foreign or modified gene or polynucleotide sequence that is artificially introduced into the genome of another organism. An “adeno-associated virus capsid, particle, or vector” or “AAV capsid, particle, or vector” refers to a transgene packaged in a viral capsid comprising viral proteins VP1, VP2, and VP3. Techniques and methods for AAV vector generation are known in the art. Generally, AAV vector production utilizes (a) a plasmid containing the AAV rep and cap genes for capsid formation and replication; (b) a plasmid containing adenovirus helper genes; (c) a cassette containing a target transgene flanked by inverted terminal repeats; and (d) a viral packaging cell lines such as HEK293 cells. As one of ordinary skill will appreciate, the methods and applications disclosed herein can be applied to any AAV, regardless of the techniques, methods, materials, and protocols used to generate them.

In a further aspect, the present disclosure relates to a method for performing cIEF to separate and characterize full viral vectors, partial viral vectors, and empty viral vectors. In specific embodiments, a cIEF method is used to separate and characterize AAV capsids, particles, or vectors as having a full transgene and promoter, a transgene fragment, or lacking a transgene. cIEF utilizes zwitterionic carrier ampholytes to create a pH gradient within the capillary. Carrier ampholytes can be wide-range (e.g. pH 3-10), narrow-range (e.g. pH 6-8), or a combination thereof, though other ranges may also be used such as pH 3-10 or pH 7-10. As with CE, a first end of the capillary is fluidly connected to a basic solution while the second end is fluidly connected to an acidic solution.

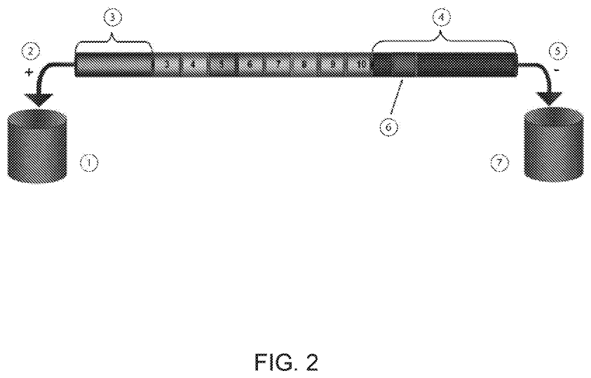

FIG. 2 illustrates an exemplary embodiment of a cIEF setup that includes anolyte solution 1, anode 2, anodic stabilizer 3, cathodic stabilizer 4, cathode 5, detection window 6, and catholyte solution 7. As voltage is applied to the capillary, a pH gradient forms from hydronium ions supplied from anolyte solution 1 and hydroxyl ions from catholyte solution 2. During cIEF focusing, cathodic stabilizer 4 migrates toward cathode 5, while anodic stabilizer 3 migrates toward anode 2 (Cruzado-Park, I. D., Mack, S., and Ratnayake, C. K., Application Information Bulletin A-11634A: Identification of System Parameters Critical for High Performance cIEF, Beckman Coulter, Inc., Fullerton, Calif., 2008, incorporated by reference herein in its entirety). Focusing is bi-directional with the pH gradient forming at both capillary ends, and progressing toward the center of the capillary such that a continuous pH gradient is created (Hjerten, S., Liao, J. L., and Yao, K. Q., J Chromatogr, Volume 387, pp 127, 1987, incorporated by reference herein in its entirety). Sample components or analytes of interest migrate and separate in the pH gradient based on their isoelectric point (pI). Once an analyte achieves a zero net charge, it ceases migration.

After pH gradient formation and focusing of sample analytes, mobilization occurs. During cIEF mobilization pI standards or markers and focused analytes of interest are detected. Mobilization can be accomplished in either the cathodic or anodic direction. For cathodic mobilization, the cathode reservoir is filled with a mobilizing solution such as acetic acid or a solution of sodium hydroxide and sodium chloride (Manabe, T., Miyamoto, H., and Iwasaki, A., Electrophoresis, Volume 18, pp 92, 1997 and Application Information Bulletin A-12015A: A Robust cIEF Method: Intermediate Precision for the pH 5-7 Range, Beckman Coulter, Inc., Fullerton, Calif., 2008, incorporated by reference herein in their entireties). For anodic mobilization, the anode reservoir is filled with a mobilizing solution such as sodium chloride. When voltage is applied during cathodic mobilization, hydronium ions are introduced into the capillary from the anolyte, while acetate ions are introduced from the cathodic side. In other embodiments, chloride and hydroxide ions are introduced. This results in titration of the pH gradient from basic to acidic, and separated analytes are detected as they obtain a positive charge and migrate toward the cathode. The detection wavelength is selected based on the detector. In some embodiments, a UV detector and a 280 nm filter are used.

In one aspect, the methods disclosed herein can separate and distinguish viral vectors as having a full length exogenous polynucleotide, having an exogenous polynucleotide fragment, or lacking any exogenous polynucleotide, based on the charge heterogeneity or pI of each viral vector. Specifically, the readout or results from CE, including cIEF, can be used to identify viral vectors by exogenous polynucleotide signature, which is determined by the migration pattern and resulting electropherogram peak.

In another aspect, the cIEF method disclosed herein can separate and distinguish viral vectors as having a full length exogenous polynucleotide, having an exogenous polynucleotide fragment, or lacking any exogenous polynucleotide, based on the pI of each viral vector. Specifically, in the resulting cIEF electropherogram or separation scan (e.g., whole-column imaging detection (WCID)), peaks represent full, partial, and empty viral vectors. Empty viral vectors migrate at an alternate pI, while full viral vectors can migrate at a lower pI. Peaks between “empty” and “full” vectors indicate partial viral vectors (i.e. those containing exogenous polynucleotide fragments). In this respect, the cIEF method can determine the pI of the viral vector, which can subsequently be used to identify full, partial, and empty vectors.

In a further aspect, the cIEF method disclosed herein can separate and distinguish AAV capsids, particles, or vectors as having a full genome with transgene and promoter, a transgene fragment, or lacking a transgene, based on the pI of each AAV capsid. cIEF migration peaks of empty AAV capsids occur at an alternate pI relative to the viral vectors having an exogenous polynucleotide or fragment thereof, or viral vectors lacking any exogenous polynucleotide, while in some embodiments migration peaks of full AAV capsids may occur, relative to empty capsids at a lower pI, with migration peaks of partial AAV vectors falling in between. Typically, full AAV capsids have a lower pI due to the negative charge of the exogenous polynucleotide encapsulated within the viral capsid.

In some embodiments, the pI of an AAV capsid containing a full exogenous polynucleotide or transgene is about 0.3-0.5 pI units lower than an AAV capsid lacking an exogenous polynucleotide or transgene. In some embodiments the difference in pI may be less than 0.1 pH unit and, in such embodiments, the ampholyte may be modified (e.g., an ampholyte having a narrow pH range) in order to improve resolution of capsids that have small differences in pI.

In yet another aspect, the relative abundance of full, partial, and empty viral vectors in a sample comprising multiple viral vectors can be determined via quantification of peak area. For example, a sample enriched in empty viral vectors will have a larger peak area at an “empty” viral vector pI, while a sample enriched in full viral vectors will have a larger peak area at a “full” viral vector pI.

In yet another aspect, the relative abundance of full, partial, and empty AAV capsids, particles, or vectors in a sample containing multiple AAV capsids can be determined via quantification of peak area. A sample enriched in empty AAV capsids will have a larger peak area at an “empty” AAV capsid pI, while a sample enriched in full AAV capsids will have a larger peak area at a “full” AAV capsid pI. Partial AAV capsids can be calculated from the peak area between empty and full AAV capsid peaks.

Advantages of the disclosed methods include high-throughput separation within a relatively short period of time (e.g. one hour) and automated data analysis compared to analytical ultracentrifugation (AUC) and electron microscopy (EM), and minimal sample volume (e.g. 2-4 μL of vector material) compared to HPLC, AUC, and EM. Further, the methods offer superior separation and resolution as compared to anion-exchange HPLC (AEX-HPLC) and EM, and can be implemented at various stages of AAV research and development.

cIEF can be performed on any suitable CE device, for example a Sciex PA800 Plus instrument with UV detector and 214 nm filter. The capillary utilized can be a 30 cm N-CHO coated capillary. The separation voltage can be 10 kV reverse polarity. UV detection is performed at 214 nm. The buffer solution is 50 mM sodium borate at pH 8.0 and Injection is performed at

### EXAMPLES

### Example 1: Preparation of cIEF Solutions

Solutions for cIEF were Prepared as Follows:

Anolyte solution: to prepare 200 mM phosphoric acid, 0.685 mL 85% phosphoric acid was added to a total volume of 50 mL with deionized (DI) water.

Catholyte solution: to prepare 300 mM sodium hydroxide, 15 mL 1 M NaOH (Sigma, Cat #720820) was added to a total volume of 50 mL with DI water.

Chemical mobilizer solution: to prepare 350 mM acetic acid, 1 mL glacial acetic acid was added to a total volume of 50 mL DI water.

Cathodic stabilizer solution: to prepare 500 mM L-arginine, 0.87 g of 98% L-arginine (Sigma, Cat # A5006) was dissolved in 8 mL DI water and mixed for 15 min for complete solvation. The resulting solution was scaled up to a total volume of 10 mL DI water.

Anodic stabilizer solution: to prepare 200 mM of iminodiacetic acid (IDA), 0.27 g of 98% IDA (Sigma, Cat #220000) was dissolved in 8 mL DI water and mixed for 15 min for complete solvation. The resulting solution was scaled up to a total volume of 10 mL DI water. cIEF gel solution (U-gel): to prepare a 3 M Urea cIEF gel solution, 1.8 g of urea (Sigma, Cat # U1250) was dissolved in 7 mL cIEF gel (SCIEX, Cat #477497). Once dissolved, the solution was scaled up to a total volume of 10 mL with cIEF gel, mixed for 15 min, and filtered using a 5 μm syringe filter. The 3 M Urea-cIEF gel solution was degassed at 2000 RCF with an Allegra X-15R centrifuge (Beckman Coulter, Cat #392933) and stored at 2-8° C.

### Example 2: Sample Preparation

A master mix solution was prepared by mixing 200 μL of 3M urea-cIEF gel solution, 12 μL of ampholytes, 20 μL of cathodic stabilizer, 2 μL anodic stabilizer, 2 μL of each pI marker. Samples were prepared according to Tables 1 and 2. Serotypes 1 and 2 were concentrated to 2 mg/mL using Amicon Ultra 0.5 mL Centrifugal Filters (EMD Millipore, Cat # UFC501096). The master mix for samples 1-3 was prepared using Pharmalyte 3-10 carrier ampholytes (GE Healthcare Life Sciences, Cat #17045601). The master mix for samples 4-6 was prepared using an ampholyte mixture of Pharmalyte 3-10 carrier ampholytes and Servalyte 6-8 carrier ampholytes (Serva, Cat #42906.04) at a ratio of 4:2. Sample 6 was also analyzed using only wide range Pharmalyte 3-10 carrier ampholytes.

TABLE 1

| Serotype | Serotype Identity | Source |
|---|---|---|
| 1 | AAV-1 (empty capsids) | SCIEX |
| 2 | AAV-2 (full capsids) | SCIEX |
| | AAV5-CMV-GFP | SignaGen Laboratories, |
| 3 | (titer: 1 × 10¹³ GC/mL) | Cat # SL100819 |
| 4 | pAV-CMV-GFP (empty capsids; | Vigene Biosciences, |
| | titer: 5.1 × 10¹² GC/mL) | Lot # 2019.09.12 |
| 5 | pAV-CMV-GFP (full capsids; | Vigene Biosciences, |
| | titer: 1.1 × 10¹³ GC/mL) | Lot # 2019.09.12 |
| 6 | AAV9-CMV-GFP | SignaGen Laboratories, |
| | (titer: 3.12 × 10¹³ GC/mL) | Cat # SL100840 |

TABLE 2

| Sample | Serotype Vol (μL) | Master Mix Vol (μL) |
|---|---|---|
| 1 | 10 | 240 |
| 2 | 10 | 240 |
| 3 | 3 | 24 |
| 4 | 3 | 24 |
| 5 | 3 | 24 |
| 6 | 3 | 24 |

### Example 3: Sample Analysis

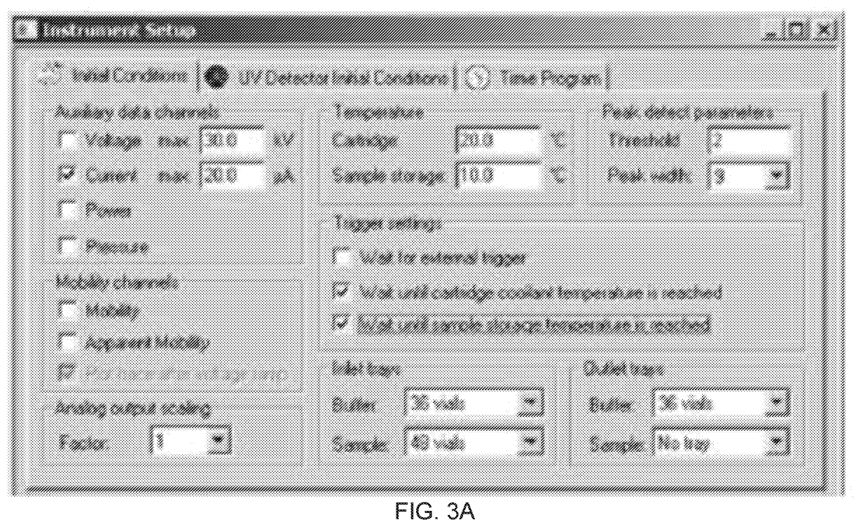

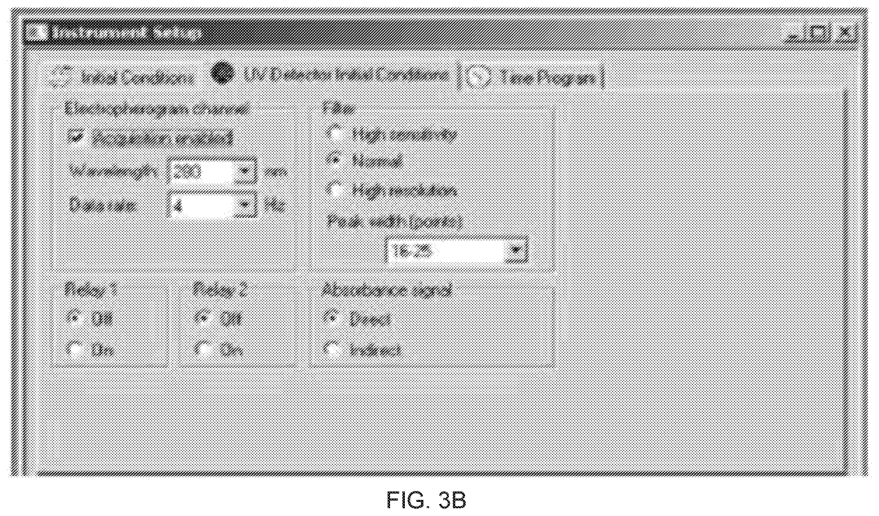

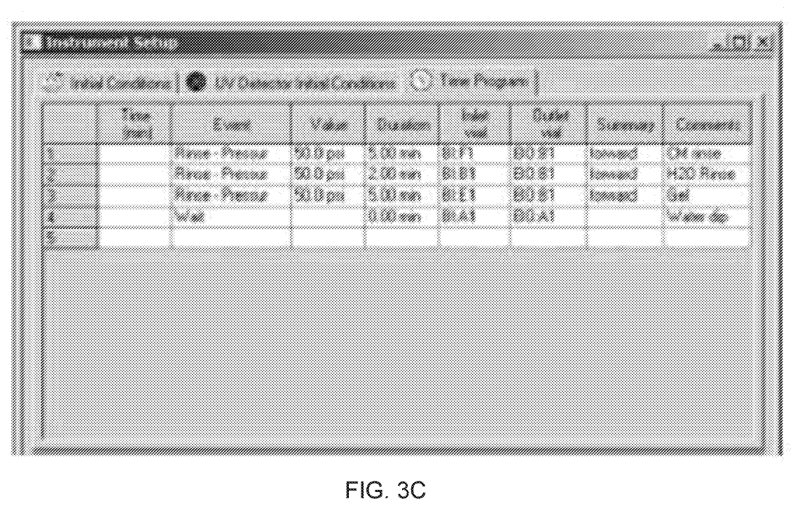

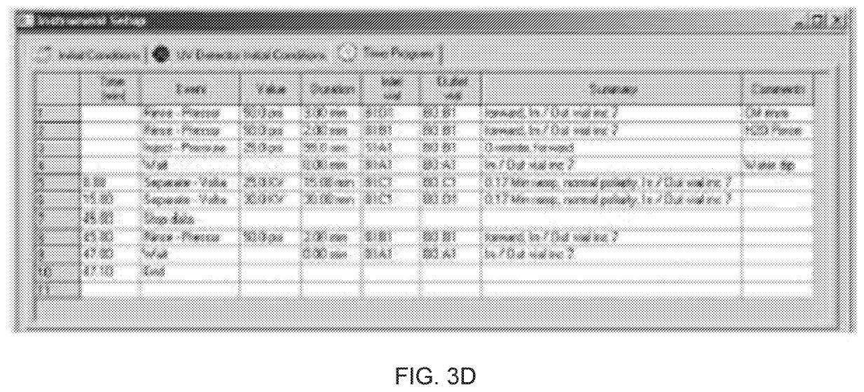

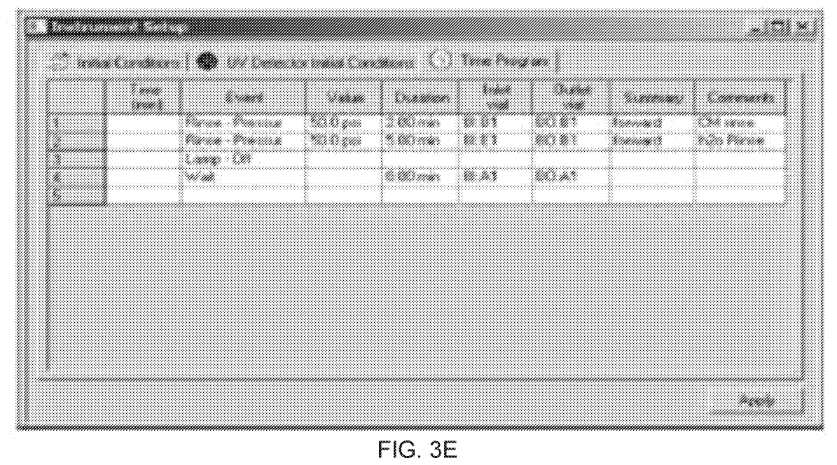

Each prepared sample was transferred to a nanoVial (SCIEX, Cat #5043467). cIEF analysis was performed using a PA 800 PA Plus Pharmaceutical Analysis System (SCIEX) equipped with a UV detector and a 280 nm filter (SCIEX, Cat #969136) and a 30.2 cm N-CHO capillary (SCIEX, Cat #477601). Instrumental parameters for the cIEF method are illustrated in FIGS. 3A-3E. Capillary temperature was maintained at 20° C. in all separations. Data were collected and analyzed using 32 Karat Software.

### Example 4: cIEF Resolution of Full, Empty, and Partial AAV Capsids

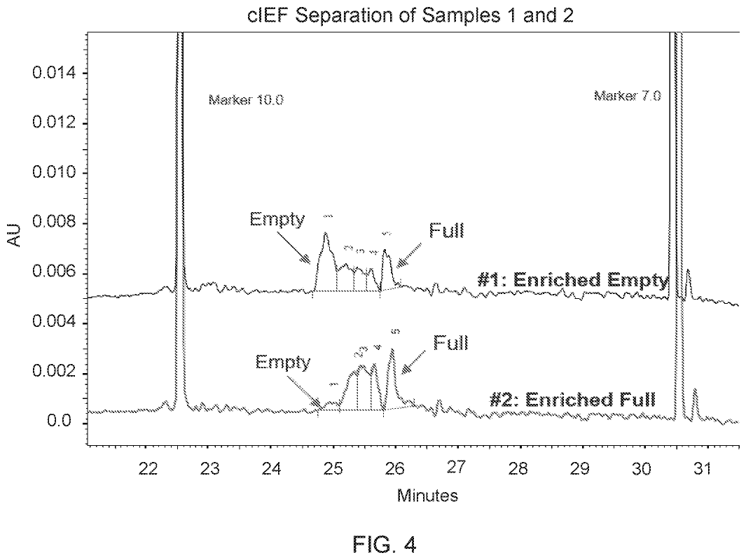

FIG. 4 illustrates the cIEF profiles for sample 1 (enriched with empty AAV capsids) and sample 2 (enriched with full AAV capsids) between pI markers 7.0 and 10.0. The empty capsid peak migrated at a higher pI while the full capsid peak migrated at a lower pI value. Peaks between the “empty” and “full” capsid peaks indicate partial capsids. As expected, sample 2 exhibited a higher intensity full capsid peak while sample 1 exhibited a higher intensity empty capsid peak. These cIEF profiles were consistent with profiles obtained by analytical ultracentrifugation (data not shown). The pI of peaks 1-5 in FIG. 4 are shown in Table 3. pI values of the AAV capsid peaks were determined based on the calibration curve of internal pI markers.

TABLE 3
pI of Separated Peaks in Samples 1 and 2

| | |
|---|---|
| Sample 1 | Sample 2 |

| Capsid | Peak | pI | Peak | pI |
|---|---|---|---|---|
| Empty | 1 | 9.09 | 1 | 9.11 |
| Partial | 2 | 8.95 | 2 | 8.99 |
| | 3 | 8.92 | 3 | 8.92 |
| | 4 | 8.84 | 4 | 8.84 |
| Full | 5 | 8.73 | 5 | 8.73 |

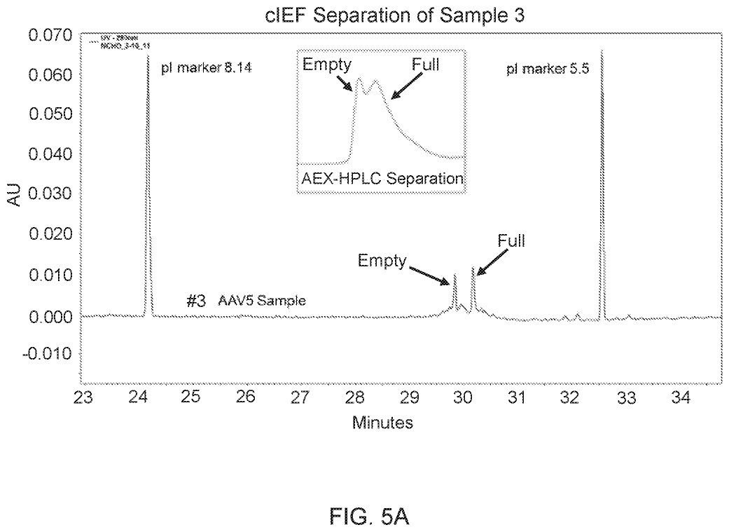

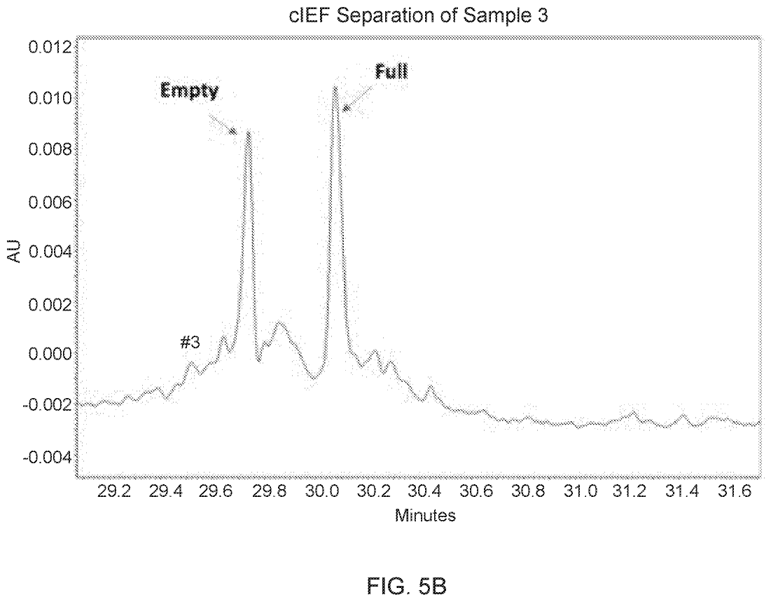

FIG. 5A illustrates the cIEF profile for sample 3 as well as the AEX-HPLC profile (inset in FIG. 5A). FIG. 5B is an enlarged view of the sample peaks from FIG. 5A. Consistent with samples 1 and 2 (discussed above), the empty capsid peak migrated at a higher pI as compared to the full capsid peak. When compared to AEX-HPLC separation, cIEF separation resulted in improved resolution of empty and full capsid peaks. Further, cIEF separated partial capsids while AEX-HPLC did not. Quantification of full, empty, and partial capsids following cIEF or AEX-HPLC separation is shown in Table 4. Since AEX-HPLC was unable to resolve partial capsids, the sum of full and partial capsid peak areas in the cIEF analysis was compared to the full capsid peak area from AEX-HPLC. These data demonstrate that the percentages of full and empty capsids determined by cIEF are comparable to those determined by AEX-HPLC.

TABLE 4
Comparison of Percent Peak Area of Sample 3 Full and Empty
Capsids as Determined by cIEF and AEX-HPLC Separation

| Separation Method | Empty Capsids (% area) | Full Capsids (% area) |
|---|---|---|
| cIEF | 33 | 67 (Full + Partial) |
| AEX-HPLC | 31 | 69 (Full) |

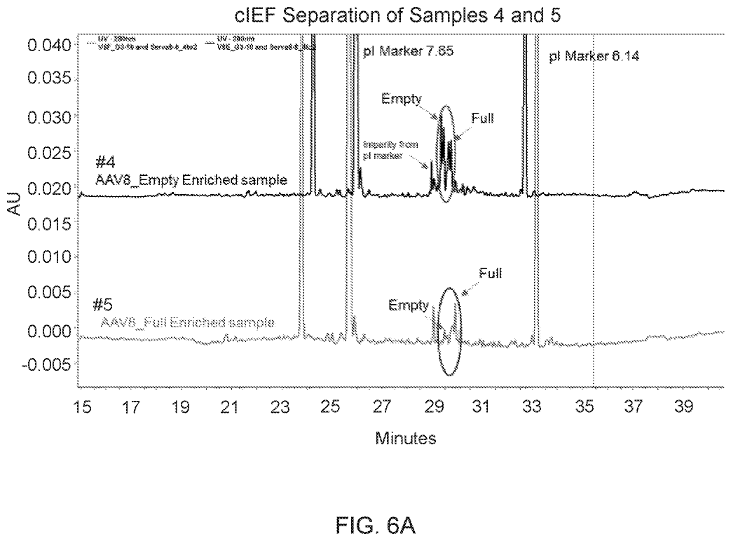

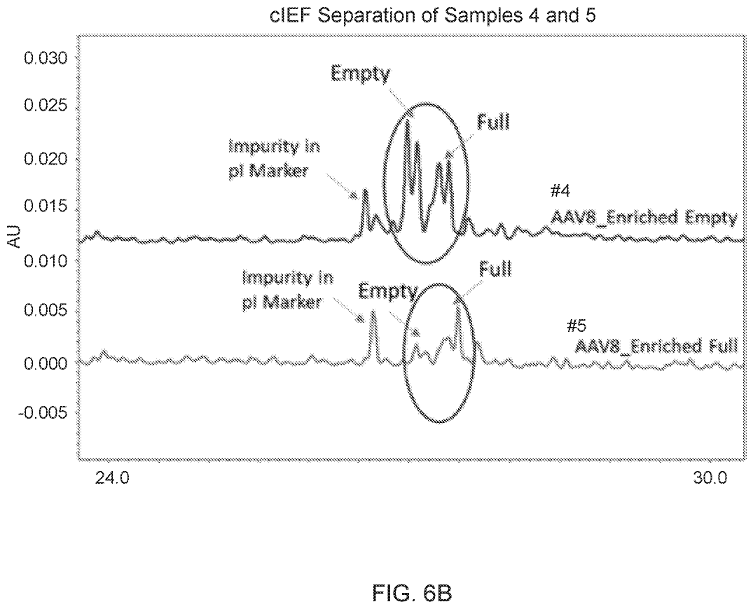

FIG. 6A illustrates the cIEF profiles for sample 4 (enriched with empty AAV capsids) and sample 5 (enriched with full AAV capsids). FIG. 6B is an enlarged view of the sample peaks from FIG. 6A. As expected, sample 5 exhibited a higher intensity full capsid peak while sample 4 exhibited a higher intensity empty capsid peak. The pI difference for samples 4 and 5 was calculated to be about 0.1 pH unit between the full and empty capsid peaks, and narrow pH range ampholytes provided superior baseline resolution of the AAV peaks. The pI value of sample 4 was approximately 7.1 (data not shown). Quantification of full, empty, and partial capsids following cIEF separation is shown in Table 5. These percentages were calculated from the corrected peak areas of the separated capsid peaks in the cIEF electropherograms.

TABLE 5
Comparison of Percent Peak Area of Full and Empty
Capsids in Samples 4 and 5 as Determined by cIEF

| Sample | Empty Capsids (% area) | Full Capsids (% area) |
|---|---|---|
| 4 | 57 | 43 |
| 5 | 22 | 78 |

### Example 5: cIEF Resolution of AAV Capsids Using Wide and Narrow Range pH Ampholytes

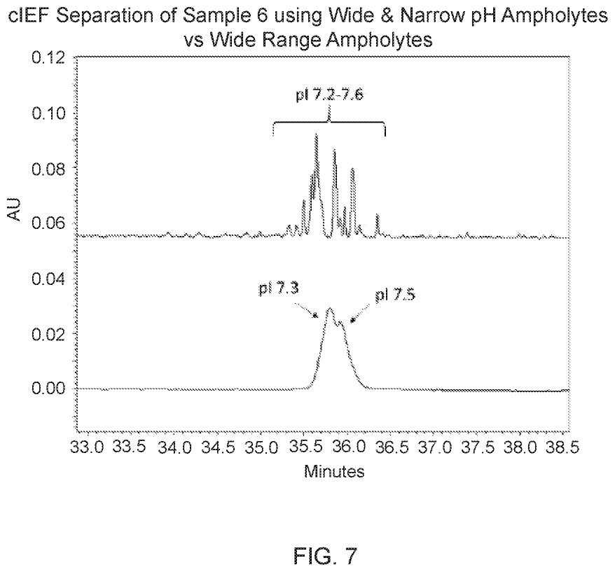

FIG. 7 compares the cIEF profiles of sample 6 using a mixture of wide and narrow range pH ampholytes (top graph) and wide range pH ampholytes (bottom graph). The wide range pH ampholytes resulted in lesser resolved peaks at pI 7.3 and 7.5, while the mixture of pH ampholytes provided enhanced sample resolution as seen in the peaks between pI7.3 and 7.6. These data demonstrate use of narrow range or narrow and wide range pH ampholytes improves resolution of partial capsid variants in AAV samples.

### Example 6: Reproducibility of cIEF Analysis of AAV Capsids

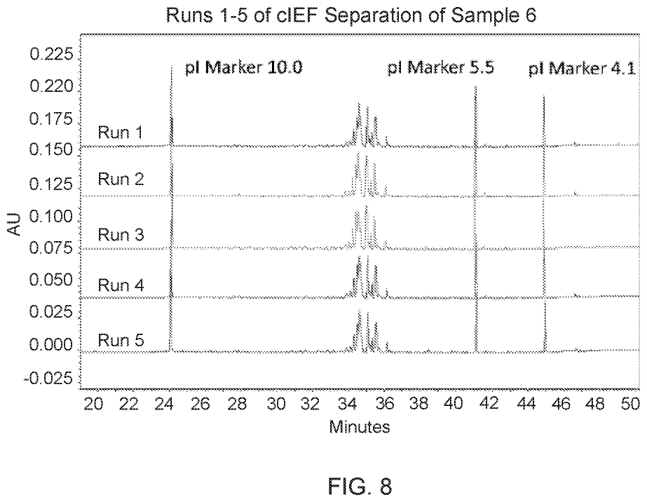

FIG. 8 shows the reproducibility of cIEF separation for an AAV sample. Five runs of cIEF were performed on sample 6. The % relative standard deviation (RSD) for peak area was <5% and the % RSD for pI value was <2%. These data demonstrate cIEF analysis of AAV capsids has a high degree of reproducibility.

Collectively, the above exemplary data demonstrates cIEF analysis can be used for differentiation and identification of AAV capsids. Specifically cIEF can identify and quantify capsids having a full transgene, having a partial transgene, or lacking a transgene. In a heterogeneous AAV sample, cIEF can determine the distribution of full, empty, and partial capsids within the sample.

### Example 7: CZE Resolution of Full and Empty AAV Capsids

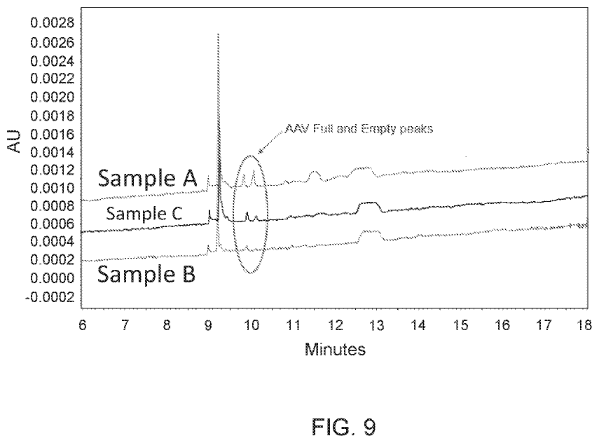

FIG. 9 illustrates the CZE profiles for sample A (enriched with empty AAV capsids), sample B (enriched with full AAV capsids), and sample C (1:1 ratio of samples A and B). The sample was used as received. 10 uL of the original sample was pipetted into a Nano Vial and loaded onto the instrument. Sample was introduced into PA800 Plus instrument with UV detector and 214 nm filter. Run parameters: Capillary: 30 cm N-CHO coated capillary, Separation: 10 kV reverse polarity, UV detection: 214 nm Buffer: 50 mM sodium borate pH 8.0, Injection: 0.5 psi for 90s. The overlaid figure are CZE analysis of AAV serotype 9 samples of 3 different mixtures, one in which the sample has been enriched with empty capsids, one in which a sample has been enriched with full capsids and one in which the full and empty capsids are mixed in a ratio of 1:1. As shown, there is a clearly evident peak at around 10.4 minutes that is more pronounced in the empty capsid sample than it is in the full capsid sample.

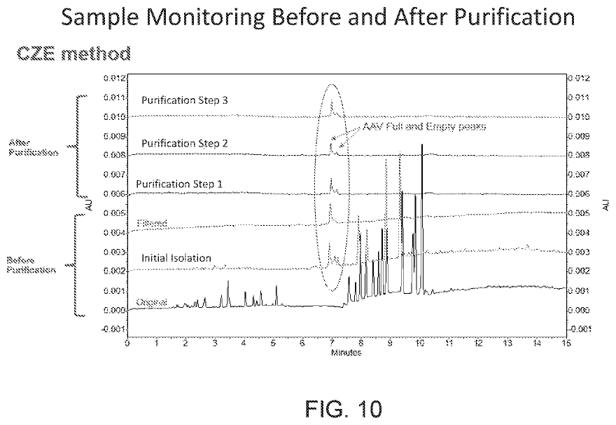

FIG. 10 illustrates the CZE profiles for an AAV sample, before and after purification. The sample was used as received. Electrokinetic injection was used to introduce the sample into the CE instrument (PA 800 Plus with PDA detector). The injection condition was at 0.5 psi for 90s and the run buffer is 50 mM sodium Borate Buffer, pH 8.5. Capillary was bare fused silica capillary with 50 μm ID and 30 cm length, the length between inlet to detection window is 20 cm. The separation condition is 10 kV reverse polarity. The overlaid traces are CZE analysis of AAV samples at each purification step in order to monitor the purity of the product as well as ratio of the full and empty capsids during the upstream development process. With each stage of the purification process, less impurity peaks are observed.

1. A method of separating viral vectors in a sample, the method comprising:
performing capillary isoelectric focusing (cIEF) on a sample comprising viral vectors containing exogenous polynucleotide under conditions and for a duration sufficient to separate the viral vectors based on the isoelectric point (pI) of each viral vector;
generating a readout of cIEF results; and
analyzing the readout to identify viral vectors based on exogenous polynucleotide content.

2. The method of claim 1, wherein the analyzing characterizes the viral vectors as having an exogenous polynucleotide comprising a full length exogenous polynucleotide, an exogenous polynucleotide fragment, or lacking any exogenous polynucleotide, wherein the viral vectors having the full length exogenous polynucleotide have a lower pI relative to the viral vectors having the exogenous polynucleotide fragment and the viral vectors lacking any exogenous polynucleotide.

3. The method of claim 1, wherein the exogenous polynucleotide comprises a transgene, promoter and ITR sequence.

4. The method of claim 1, wherein the viral vector is an adeno-associated virus vector.

5. The method of claim 1, wherein the readout of cIEF results is an electropherogram or a separation scan.

6. The method of claim 1, wherein performing cIEF comprises the steps of:
loading the sample comprising viral vectors into a channel or a capillary comprising a pH gradient;
fluidly connecting a first end of the channel or capillary to an acidic solution;
fluidly connecting a second end of the channel or capillary to a basic solution; and
applying an electric voltage to the channel or capillary.

7. The method of claim 6, wherein the channel or capillary is loaded with 2-4 μL of a vector material that comprises the sample.

8. A method of quantifying viral vectors in a sample, the method comprising:
performing capillary isoelectric focusing (cIEF) on a sample comprising viral vectors containing exogenous polynucleotide under conditions and for a duration sufficient to separate the viral vectors based on the isoelectric point (pI) of each viral vector;
generating a readout of cIEF results; and
analyzing the readout to identify viral vectors based on exogenous polynucleotide content.

9. The method of claim 8, wherein the analyzing comprises quantifying from the readout a relative abundance of viral vectors having a full length exogenous polynucleotide, viral vectors having an exogenous polynucleotide fragment, or viral vectors lacking any exogenous polynucleotide, wherein the viral vectors having the full length exogenous polynucleotide have an altered pI relative to the viral vectors having the exogenous polynucleotide fragment and the viral vectors lacking any exogenous polynucleotide.

10. The method of claim 8, wherein the exogenous polynucleotide comprises a transgene, promoter and ITR sequence.

11. The method of claim 8, wherein the viral vector is an adeno-associated virus vector.

12. The method of claim 8, wherein the readout of cIEF results is an electropherogram or a separation scan.

13. The method of claim 8, wherein performing cIEF comprises the steps of:
loading the sample comprising viral vectors into a channel or a capillary comprising a pH gradient;
fluidly connecting a first end of the channel or capillary to an acidic solution;
fluidly connecting a second end of the channel or capillary to a basic solution; and
applying an electric voltage to the channel or capillary.

14. The method of claim 13, wherein the channel or capillary is loaded with sample comprising 2-4 μL of a vector material.

15. A method of separating viral vectors in a sample, the method comprising:
performing capillary electrophoresis (CE) of a sample comprising viral vectors containing exogenous polynucleotide under conditions and for a duration sufficient to separate the viral vectors based on the charge heterogeneity or isoelectric point (pI) of each viral vector;
generating a readout of CE results; and
analyzing the readout to identify viral vectors based on exogenous polynucleotide content.

16. The method of claim 15, wherein the analyzing characterizes the viral vectors as having an exogenous polynucleotide comprising a full length exogenous polynucleotide, an exogenous polynucleotide fragment, or lacking any exogenous polynucleotide.

17. The method of claim 15, wherein the exogenous polynucleotide comprises a transgene, promoter and ITR sequence.

18. The method of claim 15, wherein the viral vector is an adeno-associated virus vector.

19. The method of claim 15, wherein the readout of CE results is an electropherogram or a separation scan.

20. The method of claim 15, wherein performing CE comprises the steps of:
loading the sample comprising viral vectors into a channel or a capillary;
fluidly connecting a first end of the channel or capillary to an acidic solution;
fluidly connecting a second end of the channel or capillary to a basic solution; and
applying an electric voltage to the channel or capillary.

21. The method of claim 20, wherein CE is selected from the group consisting of capillary isoelectric focusing (cIEF), capillary zone electrophoresis (CZE), and capillary gel electrophoresis (CGE).

22. A method of quantifying viral vectors in a sample, the method comprising:
performing capillary electrophoresis (CE) of a sample comprising viral vectors containing exogenous polynucleotide under conditions and for a duration sufficient to separate the viral vectors based on the charge heterogeneity or isoelectric point (pI) of each viral vector;
generating a readout of CE results; and
analyzing the readout to identify viral vectors based on exogenous polynucleotide content.

23. The method of claim 22, wherein the analyzing comprises quantifying from the readout a relative abundance of viral vectors having an exogenous polynucleotide comprising a full length exogenous polynucleotide, viral vectors having an exogenous polynucleotide fragment, or viral vectors lacking any exogenous polynucleotide.

24. The method of claim 22, wherein the exogenous polynucleotide comprises a transgene, promoter and ITR sequence.

25. The method of claim 22, wherein the viral vector is an adeno-associated virus vector.

26. The method of claim 22, wherein the readout of CE results is an electropherogram or a separation scan.

27. The method of claim 22, wherein performing CE comprises the steps of:
loading the sample comprising viral vectors into a channel or a capillary;
fluidly connecting a first end of the channel or capillary to an acidic solution;
fluidly connecting a second end of the channel or capillary to a basic solution; and
applying an electric voltage to the channel or capillary.

28. The method of claim 27, wherein CE is selected from the group consisting of capillary isoelectric focusing (cIEF), capillary zone electrophoresis (CZE), capillary gel electrophoresis (CGE) and capillary isotachophoresis, micellar electrokinetic capillary chromatography and capillary electrokinetic chromatography.
